(19) United States
(12) Patent Application Publication
(10) Pub. No.: US 2014/0358245 A1
(43) Pub. Date: Dec. 4, 2014

(54) SEGMENTAL URETERAL STENT

(71) Applicant: Boston Scientific Scimed, Inc., Maple Grove, MN (US)

(72) Inventors: Timothy P. HARRAH, Cambridge, MA (US); Jianmin LI, Marlborough, MA (US)

(73) Assignee: Boston Scientific Scimed, Inc., Maple Grove, MN (US)

(21) Appl. No.: 14/282,873

(22) Filed: May 20, 2014

Related U.S. Application Data
(60) Provisional application No. 61/829,022, filed on May 30, 2013.

(51) Int. Cl. A61F 2/82

(52) U.S. Cl. CPC A61F 2/82
USPC 623/23.7

(57) ABSTRACT

Ureteral stents assembled from multiple segments and methods for using the same in body lumens are described. The ureteral stents optionally include multiple segments having different compositions or characteristics, such as radiopacity, stiffness or flexibility, and loading with therapeutic agents.

### CROSS-REFERENCE TO RELATED APPLICATION

This application claims the benefit of priority under 35 U.S.C. §119(e) to U.S. Provisional Patent Application Ser. No. 61/829,022, filed May 30, 2013 and titled “Segmental Ureteral Stent.” This application is related to U.S. Pat. No. 8,021,434 by Segura et al. entitled “Ureteral Stent” (“Segura”) and to U.S. Pat. No. 6,764,519 by Whitmore III entitled “ureteral stent” (“Whitmore”). The entire disclosure of each of the foregoing references is incorporated by reference herein for all purposes

### TECHNICAL FIELD

The invention relates to medical systems and methods for facilitating fluid flows within a body, and more specifically to ureteral stents and methods of making and using the same.

### BACKGROUND

In the urinary tract, urine is conveyed from the kidney to the bladder by way of the ureter, a tubular, smooth-muscle-lined passageway. Ordinarily, urine moves one way through the ureter—from the kidney to the bladder—under the influence of hydrostatic pressure and aided by contractions of the smooth muscle lining of the ureter. When this one-way movement is disrupted,—for example by ureteral obstruction or constriction—urine may back up (reflux), leading to accumulation of urine and distension of the ureter (hydroureter), the kidney (hydronephrosis) or both the ureter and the kidney (hydorureteronephrosis), any of which may impair ureteral and/or renal function if left untreated. Common causes of ureteral obstruction or constriction include the formation of tumors or non-cancerous growths in the lining of the ureter, ureteritis and partial or total occlusion of the ureter by kidney stones, blood clots, or retroperitoneal fibrosis.

Ureteral stents are hollow, substantially tubular medical devices which extend through the ureter to facilitate drainage from kidney to the bladder when flow through the ureter is impaired. Generally, ureteral stents are made from small diameter tubing of a biocompatible plastic, and may include features to enhance drainage (e.g. multiple side holes and/or variable diameters) and improve end retention in the renal pelvis and bladder (e.g. retention hooks, pigtail curls, coils, or malecots at both proximal (bladder-side) and distal (kidney-side) ends) to prevent migration of the stent after placement.

While ureteral stents are useful in treating symptoms of ureteral obstruction or constriction, key challenges remain in the art. For one thing, ureteral stents are very useful in facilitating the flow of urine from the kidney to the bladder, but do not generally address the underlying causes of ureteral constriction or obstruction. Additionally, ureteral stents are typically pre-formed and non-adjustable, and may be uncomfortable to patients and/or not optimally effective where stent length is not well matched to patient anatomy.

### SUMMARY OF THE INVENTION

Embodiments of the current invention address the challenges described above by providing, in one aspect, a ureteral stent formed from a plurality of connectable segments. The stent may include, in some embodiments, first and second segments which have different compositions from one another. In various cases, the first segment includes a therapeutic agent that is not present in the second segment, while in other cases, the first segment includes a material having a stiffening agent that is not present in the second segment. The first and second segments are, in some embodiments, adjacent to one another, while in other embodiments they are non-adjacent. The therapeutic agent in the first segment is, variously, a solid body integrated into the first segment, or a plurality of small bodies dispersed throughout the first segment. In some cases, the second segment is radiopaque. The first and second segments are optionally characterized by different physical dimensions, including length, outer diameter, and wall thickness.

In another aspect, embodiments of the invention provide a method of treating a patient that includes the steps of forming a segmental ureteral stent by joining a plurality of segments, including at least a first segment and a second segment, and positioning a portion of the ureteral stent in the ureter of a patient. In certain embodiments, the first segment includes a therapeutic agent, which therapeutic agent is optionally selected from the group consisting of calcium channel blockers, alpha blockers, anti-cholinergic, and anti-proliferative drugs.

In yet another aspect, embodiments of the invention provide a kit for performing a medical procedure, which kit includes first and second connectable segments. In various embodiments, the first and second segments have different compositions from one another: for instance, the first segment can include a therapeutic agent that is not present in the second segment, or the first segment can include a material having a stiffening agent that is not present in the second segment. The first and second segments are, in some embodiments, adjacent to one another, while in other embodiments they are non-adjacent. The therapeutic agent in the first segment is, variously, a solid body integrated into the first segment, or a plurality of small bodies dispersed throughout the first segment. In some cases, the second segment is radiopaque. The first and second segments are optionally characterized by different physical dimensions, including length, outer diameter, and wall thickness.

### DRAWINGS

In the drawings, like reference characters refer to like features through the different views. The drawings are not necessarily to scale, with emphasis being placed on illustration of the principles of the invention.

FIG. 1 includes schematic depictions of individual segments, multiple segments assembled into a section of a ureteral stent, and a ureteral stent comprising multiple segments.

### DETAILED DESCRIPTION OF THE PREFERRED EMBODIMENTS

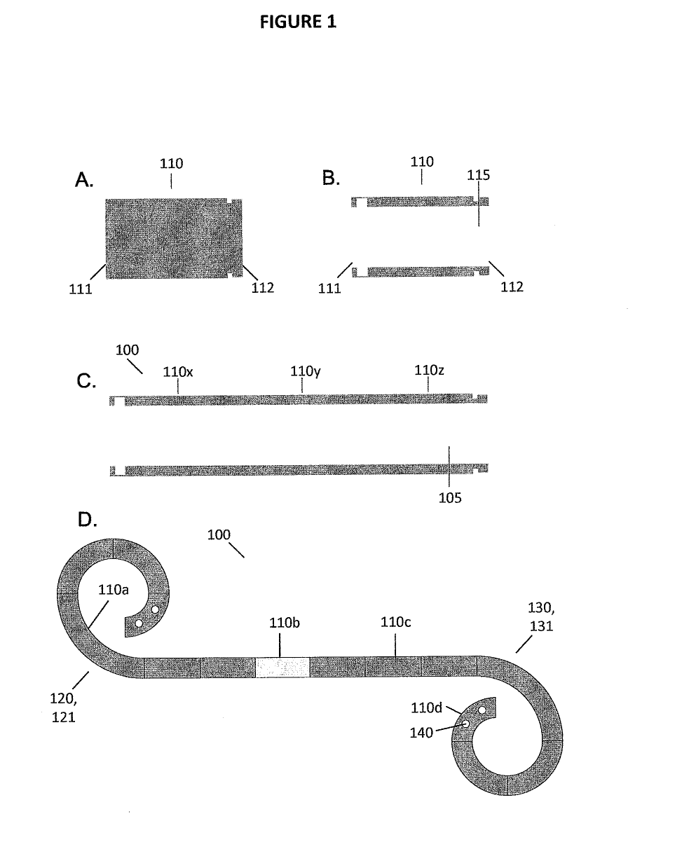

According to the embodiments shown in FIG. 1, a ureteral stent 100 of the invention is comprised of a plurality of connectable segments 110, which generally include open first and second ends 111, 112 and a segment lumen 115 extending between the first and second ends 111, 112, as shown in FIGS. 1A-B. The connectable segments 110 can be connected to one another to form extended, generally tubular structures. In particular, the individual lumens 115 of each connectable segment 110 align to form a single stent lumen 105 extending between first and second ends 120, 130 of the ureteral stent 100. The first and second ends 120, 130 of the ureteral stent 100 include one or more openings 140 to permit fluids to enter and/or exit the stent lumen 120. The first and second ends 120, 130 of the ureteral stent 100 also preferably include retention features 121, 131 to prevent migration of the ureteral stent 100 after implantation in a ureter of a patient. Exemplary retention features include pigtail coils, as shown in FIG. 1D, as well as balloons, flared portions and features described more fully in Segura and Whitmore.

Each segment 110 includes first and second ends 111, 112, which ends generally include complementary connectable structures. For instance, FIG. 1A-B shows a connector 110 that first and second ends 111, 112 that include complementary ribs and grooves to permit the first end of one connector to snap fit with a second end of another connector. Any suitable connection means for mechanically joining connectable segments 110 may be used in embodiments of the invention, including without limitation male and female screw threads, complementary tapered ends, complementary bayonet connector ends, and so on. In preferred embodiments, the connector means are adapted to achieve a mechanical fit without the need for adhesives or external mechanical fasteners, and are capable of secure attachment by application of a relatively small force—most preferably by a force less than an ordinary medical operator would apply by hand. When attached to one another, the junction between adjacent connectable segments 110 is optionally resistant to leaking or opening at or above pressures normally present in the ureter. As shown in FIG. 1C, a plurality of segments 110x, y, z are joinable to form a contiguous portion of a stent 100 including a contiguous stent lumen 105.

Segments 110 within a ureteral stent 100 of the invention are optionally characterized by variation or heterogeneity relative to one another. For instance, the composition of individual segments 110 within a ureteral stent 100 may vary. While segments 110 are generally made using materials familiar in the medical device arts, such as ethylene-vinyl acetate (EVA), polyurethane, or silicone, a first segment 110a may include one or more additives, or may otherwise vary in composition from a second segment 110b in the same ureteral stent 100. Suitable additives can include materials that alter the rigidity, flexibility or elasticity of the segment, for example reinforcing fibers or shape-memory materials which allow a segment to assume a first configuration during implantation of stent 100 and then assume a second configuration after implantation, which change in configuration can be triggered by a suitable change in temperature or force applied to the segment 110.

Another important category of additives for segments 110 according to the invention is therapeutic agents. The term therapeutic agents is used in this specification to refer to any bioactive material, including without limitation small molecule pharmaceuticals, proteins, peptides, nucleotides, lipids, sugars, fatty acids, lipids, glycolipids, glycoproteins, radioactive materials, and other materials familiar to those of skill in the art. In preferred embodiments, a first segment 110a of a ureteral stent according to the invention includes a therapeutic agent selected to treat a disease, disorder or condition that contributes to the occlusion or constriction of the ureter. For instance, where the ureter is occluded by a tumor, first segment 110a can include a chemotherapeutic selected for its efficacy in treating tumors. Similarly, where a ureter is constricted due to spasm, a calcium channel blocker, alpha-adrenoreceptor blocker, anti-cholinergic, or similar drugs may be incorporated into the first segment 110.

By including a therapeutic agent in segment 110, users are advantageously able to provide for depot release of drug, as well as for the administration of drug directly to regions in and around the ureter. These advantages, in turn, permit high concentrations of therapeutic agents to be delivered over long intervals in a localized way, potentially reducing adverse effects associated with systemic and repeated delivery of drugs that act on the urinary tract. For instance, urinary tract conditions such as tumors, fibrosis, or spasm that are currently treated by systemic administration of drugs can be treated using a segmental ureteral stent 100 of the current invention, which stent 100 includes at least one segment 110 that is loaded with an anti-proliferative or other anti-tumor drug. The stent is implanted within a patient so that the drug loaded segment or segments 110 is sufficiently proximate to the site of a tumor, disease or other condition to provide an effective dose of drug to the site. The drug loaded segment or segments 110 are also preferably loaded with a quantity of drug sufficient to provide, via diffusion or other mechanisms, an effective dose to sites proximate to the stent 100 without substantially raising a concentration of the drug in the bloodstream. In some instances the distance between the drug loaded segment or segments 110 and portions of the renal vasculature is maximized or otherwise selected to minimize any increase in concentraton of the drug in the bloodstream.

Segments 110 can also vary structurally. For instance, a first segment can be substantially straight, and can be used to form a central portion of a stent 100, while a second segment can include a retention feature or a portion thereof. In the exemplary stent of FIG. 1D, pigtail coils at the first and second ends 120, 130 of the stent 100 are formed from one or more segments 110.

In use, a plurality of segments 110 are provided, for example in a kit, to a user along with instructions for assembling the segments 110 into a stent 100. The kit preferably includes segments 110 which have varying characteristics such as drug content and concentration, length, width, internal diameter, external diameter, curvature, the presence or absence of male and/or female ends, the presence or absence of retention features and the presence or absence of openings 140. For instance, in an exemplary kit according to the invention, a segment 110a comprises all or part of a pigtail coil, while another segment 110b includes a drug effective in treating a condition contributing to the obstruction or occlusion of the ureter, another segment 110c is substantially straight and does not incorporate drug, and yet another segment 110d includes apertures 140 to permit fluid to flow into and/or out of the segment. The kit preferably includes a sufficient number of segments having different characteristics to permit a user to tailor one or more of a length, an outer dimension, an inner dimension, and a drug dosage to a user without using all of the segments provided in the kit.

A kit according to the invention also preferably includes instructions, or directions for use, instructing a user in a method of assembling the segments 110 into a ureteral stent 100. Following the instructions, the user connects the connectable segments 110 to form a ureteral stent 100, and then positions the ureteral stent within the urinary tract so that at least a part of the stent 100 resides in each of the renal pelvis, the ureter, and the bladder. Preferably, retention features at the first and second ends 120, 130 of the stent 100 are positioned in the renal pelvis and the bladder, respectively, while a middle portion of the stent 100 extends through the ureter.

The phrase “and/or,” as used herein should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “consists essentially of” means excluding other materials that contribute to function, unless otherwise defined herein. Nonetheless, such other materials may be present, collectively or individually, in trace amounts.

As used in this specification, the term “substantially” or “approximately” means plus or minus 10% (e.g., by weight or by volume), and in some embodiments, plus or minus 5%. Reference throughout this specification to “one example,” “an example,” “one embodiment,” or “an embodiment” means that a particular feature, structure, or characteristic described in connection with the example is included in at least one example of the present technology. Thus, the occurrences of the phrases “in one example,” “in an example,” “one embodiment,” or “an embodiment” in various places throughout this specification are not necessarily all referring to the same example. Furthermore, the particular features, structures, routines, steps, or characteristics may be combined in any suitable manner in one or more examples of the technology. The headings provided herein are for convenience only and are not intended to limit or interpret the scope or meaning of the claimed technology.

Certain embodiments of the present invention have been described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made express herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

What is claimed is:

1. A ureteral stent, comprising first and second connectable segments.

2. The ureteral stent of claim 1, wherein each of the first and second connectable segments includes open male and female ends and a lumen extending between the open male and female ends, wherein the male end of the first connectable segment is connected to the female end of the second connectable segment.

3. The ureteral stent of claim 1, wherein the first connectable segment has a different composition than the second connectable segment.

4. The ureteral stent of claim 3, wherein the first connectable segment includes a therapeutic agent.

5. The ureteral stent of claim 4, wherein the therapeutic agent is a solid body integrated into the first connectable segment.

6. The ureteral stent of claim 4, wherein the therapeutic agent is dispersed within a wall of the first segment.

7. The ureteral stent of claim 1, wherein the first and second connectable segments are not connected to one another.

8. The ureteral stent of claim 1, wherein the second connectable segment is radiopaque.

9. The ureteral stent of claim 1, wherein the first and second connectable segments are characterized by different first and second outer diameters, respectively.

10. The ureteral stent of claim 1, wherein the first and second connectable segments are characterized by different first and second wall thicknesses, respectively.

11. The ureteral stent of claim 1, wherein the first and second connectable segments are characterized by differing first and second lengths, respectively.

12. A method of treating a patient comprising the steps of:
providing first and second connectable segments;
assembling a ureteral stent comprising the first and second connectable segments; and
positioning a portion of the ureteral stent in a ureter of the patient.

13. The method of claim 12, wherein the first connectable segment includes a therapeutic agent.

14. The method of claim 13, wherein the therapeutic agent is selected from the group consisting of calcium channel blocker, alpha blocker, anti-cholinergic, and anti-proliferative drugs.

15. A kit for treating a patient, the kit comprising:
first and second segments connectable to form at least a portion of a ureteral stent; and
instructions for assembling the first and second segments to form a portion of the ureteral stent.

16. The kit of claim 15, wherein each of the first and second connectable segments includes open male and female ends and a lumen extending between the open male and female ends, wherein the male end of the first connectable segment is connected to the female end of the second connectable segment.

17. The kit of claim 15, wherein the first connectable segment has a different composition than the second connectable segment.

18. The kit of claim 15, wherein the first connectable segment includes a therapeutic agent.

19. The kit of claim 18, wherein the therapeutic agent is a solid body integrated into the first connectable segment.

20. The ureteral stent of claim 18, wherein the therapeutic agent is dispersed within a wall of the first segment.

21. The kit of claim 15, wherein the first and second connectable segments are not connected to one another.

22. The kit of claim 15, wherein the second connectable segment is radiopaque.

23. The kit of claim 15, wherein the first and second connectable segments are characterized by different first and second outer diameters, respectively.

24. The kit of claim 15, wherein the first and second connectable segments are characterized by different first and second wall thicknesses, respectively.

25. The kit of claim 15, wherein the first and second connectable segments are characterized by differing first and second lengths, respectively.
